(19) United States
(12) Patent Application Publication
(10) Pub. No.: US 2024/0249800 A1
(43) Pub. Date: Jul. 25, 2024

(54) TRAINED MACHINE LEARNING MODEL FOR FORECASTING MOLECULAR CONFORMATIONS

(71) Applicant: Microsoft Technology Licensing, LLC, Redmond, WA (US)

(72) Inventors: Leon Immanuel KLEIN, Berlin (DE); Yue Kwang FOONG, Cambridge (GB); Tor Erlend FJELDE, Cambridge (GB); Bruno Kacper MLODOZENIEC, Cambridge (GB); Marc Manuel Johannes BROCKSCHMIDT, Cambridge (GB); Reinhard Sebastian Bernhard NOWOZIN, Cambridge (GB); Frank NOE, Berlin (DE); Ryota TOMIOKA, Cambridge (GB)

(73) Assignee: Microsoft Technology Licensing, LLC, Redmond, WA (US)

(21) Appl. No.: 18/179,621

(22) Filed: Mar. 7, 2023

Related U.S. Application Data
(60) Provisional application No. 63/481,585, filed on Jan. 25, 2023.

(51) Int. Cl. G16C 20/70; G16C 10/00

(52) U.S. Cl. CPC G16C 20/70; G16C 10/00

(57) ABSTRACT

A computerized method for forecasting a future conformation of a molecular system based on a current conformation of the molecular system comprises (a) receiving the current conformation in a trained machine-learning model that has been previously trained to map a plurality of conformations received to a corresponding plurality of conformations proposed; (b) mapping the current conformation to a proposed conformation via the trained machine-learning model, wherein the proposed conformation is appended to a Markov chain; and (c) returning the proposed conformation as the future conformation.

### CROSS-REFERENCE TO RELATED APPLICATION

This application claims priority to U.S. Provisional Patent Application Ser. No. 63/481,585 filed 25 Jan. 2023 and entitled TRAINED MACHINE LEARNING MODEL FOR FORECASTING MOLECULAR CONFORMATIONS, the entirety of which is hereby incorporated herein by reference for all purposes.

### BACKGROUND

Molecular dynamics (MD) is a method for simulating the movements of atoms in molecules. The method integrates, in effect, the equations of motion for a system of interacting particles, to predict the time evolution of the nuclear coordinates of a chemical system based on a given set of initial nuclear coordinates. The results of MD simulation can aid the understanding of chemical reactions (including kinetics), as well as phase transitions and conformational changes, including guest-host binding and protein folding.

### SUMMARY

One aspect of this disclosure relates to a computerized method for forecasting a future conformation of a molecular system based on a current conformation of the molecular system. The method comprises (a) receiving the current conformation in a trained machine-learning model that has been previously trained to map a plurality of conformations received to a corresponding plurality of conformations proposed; (b) mapping the current conformation to a proposed conformation via the trained machine-learning model, wherein the proposed conformation is appended to a Markov chain; and (c) returning the proposed conformation as the future conformation.

Another aspect of this disclosure relates to a computer system for forecasting a future conformation of a molecule based on a current conformation of the molecule, wherein at least the future conformation is a sample drawn from a Boltzmann distribution of conformations of the molecule. The computer system comprises a processor and associated computer memory storing a molecular dynamics program that, when executed, causes the processor to implement certain computational engines. The engines include: (a) an input engine configured to receive a primary structure of the molecule; (b) a generator engine configured to generate the current conformation of the molecule based on the primary structure received; (c) an MD-accelerator engine, and (d) an output engine. The molecular dynamics accelerator engine is configured to (i) receive the current conformation in a trained machine-learning model that has been previously trained to map a plurality of conformations received to a corresponding plurality of conformations proposed; (ii) map the current conformation to a proposed conformation via the trained machine-learning model, wherein the proposed conformation is appended to a Markov chain; (iii) submit the proposed conformation to a Metropolis-Hastings test configured to accumulate the Boltzmann distribution without asymptotic bias, by accepting some of the conformations proposed and rejecting a balance of the conformations proposed; and (iv) return the proposed conformation as the future conformation if the proposed conformation is accepted by the Metropolis-Hastings test. The output engine is configured to output the future conformation of the molecule as returned by the accelerator engine.

This Summary is provided to introduce in simplified form a selection of concepts that are further described in the Detailed Description. This Summary is not intended to identify key features or essential features of the claimed subject matter, nor is it intended to be used to limit the scope of the claimed subject matter. The claimed subject matter is not limited to implementations that solve any disadvantages noted in any part of this disclosure.

### BRIEF DESCRIPTION OF THE DRAWINGS

FIG. 1 is a generalized schematic view that illustrates a high-level processing pipeline of a molecular dynamics (MD) accelerator program according to one example of the present disclosure. The pipeline may be implemented on a computer system as shown in FIG. 10 or in other suitable computing environments, such as the environment of FIG. 12.

FIG. 2 shows example pseudocode for a Markov-chain Monte Carlo (MCMC) method implemented by an MD accelerator program.

FIGS. 3A through 3C provide a schematic illustration of a conditional-flow architecture of an example MD-accelerator program.

FIGS. 4A through 4C show example results of alanine dipeptide experiments conducted using an MD accelerator program.

FIGS. 5A through 5E show example results of additional dipeptide experiments conducted using an MD accelerator program.

FIG. 6 shows example results of tetrapeptide experiments conducted using an MD accelerator program.

FIGS. 7A through 7C show example results of additional tetrapeptide experiments conducted using an MD accelerator program.

FIG. 8 shows aspects of an example method to forecast a future conformation of a molecule based on a current conformation of the molecule.

FIG. 9 shows aspects of an example method to train the machine-learning model used in the method of FIG. 8.

FIG. 10 shows aspects of an example computer system configured to execute the method of FIG. 8 and implement the MD accelerator program of FIG. 1.

FIG. 11 shows example pseudocode for a fast exploration algorithm, which may be implemented by an MD accelerator program.

FIG. 12 is a simplified block diagram of an example computer system, which can be used to implement the systems and methods described herein.

### DETAILED DESCRIPTION

Molecular dynamics (MD) is a mature technology for simulating the motion of atoms in molecules. MD works by accurately integrating the equations of motion for the atoms on the femtosecond (fs) time scale (1 fs=10⁻¹⁵ second), thereby drawing samples from the equilibrium Boltzmann distribution. This short timescale effectively bars the application of MD to processes occurring on much longer timescales, such as the millisecond timescale of biomolecular dynamics. Disclosed herein is an MD accelerator—i.e., an enhanced sampling method which uses a normalizing flow for proposal distribution in an asymptotically unbiased Markov chain targeting the Boltzmann distribution. The flow is trained offline and learns to make large steps in time, simulating molecular dynamics over very long timescales—e.g., 10⁵-10⁶ fs. Once trained, the MD accelerator can generalize to molecules outside of the training set—e.g., small molecules such as peptides (2 to 4 amino acids), providing wall-clock sampling acceleration relative to native MD. This result is an important step toward the development of accurate and generalizable deep-learning models for biomolecular dynamics.

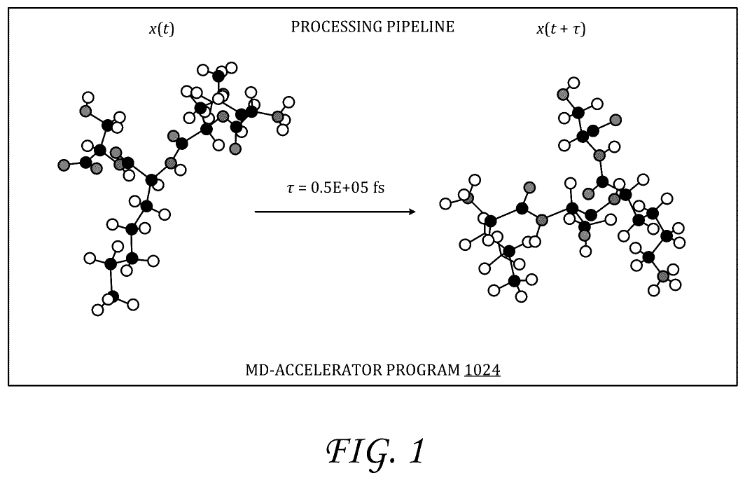

Turning now to the drawings, FIG. 1 illustrates, in one example, a high-level strategy of a processing pipeline of an MD accelerator program, as described in greater detail herein. On the leftis an initial state x(t), and on the right is a corresponding, accepted proposal state x(t+τ)˜p_{θ}(x(t+τ)|x(t)), sampled from the MD accelerator in the Markov chain for the tetrapeptide leucine-alanine-lysine-serine (LAKS). The proposed state undergoes a large rotation and conformational change relative to the initial state, traversing from one metastable state of the peptide to another.

When performed accurately, MD provides insight into the detailed mechanics of molecular motion. MD has found use in many applications, from predicting how a drug molecule binds to a protein in the human body, to understanding rates of chemical reactions. Many applications of MD boil down to efficiently sampling from the Boltzmann distribution—i.e., the equilibrium distribution of a molecular system at a given temperature T.

Let (x^{p}(t), x^{v}(t)):=x(t)∈ [symbol] be the state of the molecule at time t, including the positions x^{p}(t)∈ [symbol] and velocities x^{v}(t)∈ [symbol] of the N atoms in the molecule in Cartesian coordinates. The Boltzmann distribution is given by:

$$μ ( {x}^{p} , {x}^{v} ) = \frac{1}{Z} exp ( - \frac{1}{{k}_{B} T} ( U ( {x}^{p} ) + K ( {x}^{v} ) ) ) , \qquad ( 1 )$$

where U(x^{p}) is the potential energy, K(x^{v}) is the kinetic energy, k_{B} is Boltzmann's constant, and Z is the partition function, an intractable normalizing constant. In practice the marginal distribution of the positions, μ(x^{p}) is of greatest importance. Many important quantities in molecular biophysics, such as free energies associated with protein folding and protein-ligand binding, can be computed as expectations under the Boltzmann distribution.

Unfortunately, obtaining independent and identically distributed (i.i.d.) samples from eq 1 is extremely difficult, due to its high degree of multimodality given the presence of metastable states. One approach to sampling from eq 1 is to use MD to generate a long trajectory of atomic configurations. As the length of the trajectory increases, the configurations are asymptotically distributed according to eq 1. However, many biomolecules contain metastable states that can take on the order of milliseconds of MD simulation time to visit. This can render the sampling infeasible. In an attempt to address this challenge, prior approaches have produced a wide array of enhanced sampling methods—e.g., coarse graining and meta-dynamics, which attempt to draw samples from the Boltzmann distribution more efficiently than via MD. However, compared to MD all such enhanced sampling methods require additional domain knowledge and parameters, thereby increasing their data requirements computational complexity, and hence cost.

The MD accelerator proposed herein is a generally applicable, normalizing-flow model, which acts as a proposal distribution for a Markov-chain Monte Carlo (MCMC) method targeting the Boltzmann distribution. Unlike some prior approaches applying deep learning to protein simulation, the presently disclosed MD accelerator: (i) targets the Boltzmann distribution in an asymptotically unbiased way due to its incorporation of a Metropolis-Hastings correction, (ii) can normalize to small peptides (2 to 4 amino acids) that are not included in the training data, providing wall-clock sampling acceleration relative to native MD in terms of effective sample-size-per-second, and (iii) does not rely on internal coordinates, but works directly with an all-atom representation in Cartesian coordinates, allowing application to very general molecular configurations.

The features of this disclosure include: (a) development of the MD accelerator, a novel deep learning architecture acting on atoms via a series of atom transformer layers, for example, to define a RealNVP-based normalizing flow; (b) proposal of a new dataset of MD trajectories of small peptides to train the MD accelerator; (c) defining a sampling algorithm based on auxiliary MCMC, using the MD accelerator to generate molecular trajectories more efficiently than MD; (d) demonstrating that the MD accelerator allows wall-clock acceleration of MD sampling on small peptides absent from the training set.

MD aims to simulate the following stochastic differential equation (SDE) for the time-evolution of x^{p}(t), x^{v}(t):

$${m}_{i} d {x}_{i}^{v} = - {∇}_{i} Udt - γ {m}_{i} {x}_{i}^{v} d t + \sqrt{2 {m}_{i} γ {k}_{B} T} {dB}_{t} . \qquad ( 2 )$$

Here i=1, . . . , N indexes the atoms, mᵢ is the mass of atom i, U(x^{p}) is the potential energy, y is a friction parameter, and dBₜ is a standard Brownian motion process. Eq 2 is known as Langevin dynamics, and represents Newton's laws of motion in the presence of thermal noise. Starting from an initial state x(0), simulating eq 2, along with the relationship dx^{p}=x^{v}dt, yields values of x(t) that are distributed according to the Boltzmann distribution as t→∞. Hence sampling from the equilibrium Boltzmann distribution can be achieved by simulating Langevin dynamics for a sufficiently long time.

In practice, it is not feasible to exactly simulate a continuous-time SDE such as eq 2. Hence, standard MD libraries discretize eq 2 with a finite integration timestep Δt, typically using a stochastic version of Verlet integration (also known as the leapfrog method). For the simulation to be stable, Δt is usually chosen to be on the order of 1 fs=10⁻¹⁵ s. Unfortunately, this can render sampling from the equilibrium Boltzmann distribution infeasible, as processes that describe the large conformational changes required to explore the various metastable states of biologically interesting molecules can often take on the order of milliseconds. Thus, obtaining unbiased samples from the Boltzmann distribution may require ˜10¹² integration steps.

To address this issue, an MD accelerator is disclosed herein. The MD accelerator is a deep probabilistic model for the distribution of x(t+τ) given x(t), where τ>>Δt. Once trained, this model can be used as part of an MCMC method to efficiently sample from the Boltzmann distribution. In detail, consider the distribution of x(t+τ) induced by an MD simulation of eq 2 for a time t, starting from an initial state x(t). Due to the randomness in the Langevin dynamics, simulating eq 2 leads to a distribution of possible values for x(t+τ). This conditional distribution is denoted u(x(t+τ)|x(t)). A sample from this distribution can be obtained by initializing the MD trajectory at x(t) and integrating (a discretization of) eq 2 for τ/Δt timesteps until the time t+τ is reached.

The exact form of the conditional distribution is analytically intractable. To approximate it, a conditional normalizing-flow model, p_{θ}(x(t+τ)|x(t)), is fit to u(x(t+τ)|x(t)), where θ are learnable parameters (e.g., neural-network weights). Normalizing flows are a family of distributions defined by a simple base distribution (usually a standard Gaussian), and a diffeomorphism ƒ (i.e., a differentiable bijection with a differentiable inverse). Specifically, p_{θ}(x(t+τ)|x(t)) is set to the density of the distribution defined by the following generative process:

$$\begin{matrix}{z}^{p} , {z}^{v} ∼ 𝒩 ( 0 , I ) \\ {x}^{p} ( t + τ ) , {x}^{v} ( t + τ ) := {f}_{θ} ( {z}^{p} , {z}^{v} ; {x}^{p} ( t ) , {x}^{v} ( t ) ) .\end{matrix} \qquad ( 3 )$$

Here z⁹∈ [symbol] and z^{v}∈ [symbol] are latent variables that get transformed by the function ƒ_{θ}. For all settings of θ and the conditioning state x(t), ƒ_{θ}(·; x(t)) is a diffeomorphism that takes the latent variables (z^{p}, z^{v})∈ [symbol] to (x^{p}(t+τ), x^{v}(t+τ))∈ [symbol] . The conditioning state x(t) thus parameterizes a family of diffeomorphisms, allowing ƒ_{θ} to define a conditional normalizing flow. The function ƒ_{θ} itself is an instance of a real non-volume preserving (RealNVP) transformation, suitably modified to handle molecular inputs and conditioning states, which is described in detail below. There are no invertibility constraints on the mapping from the conditioning state x(t) to the output x(t+τ), only the map from the latent variable z to x(t+τ) is to be invertible. Using the change of variables formula, the density p_{θ}(x(t+τ)|x(t)) is evaluated analytically as:

$$𝒩 ( {f}_{θ}^{- 1} ( x ( t + τ ) ; x ( t ) ) ; 0 , I ) \vert det {𝒥}_{{f}_{θ}^{- 1} ( \cdot ; x ( t ) )} ( x ( t + τ ) ) \vert \qquad ( 3 a )$$

where ƒ_{θ}⁻¹(·; x(t)): [symbol] → [symbol] is the inverse of the diffeomorphism ƒ_{θ}(·; x(t)), and [symbol] _{(·;x(t))} (x(y+τ) denotes the Jacobian of ƒ_{θ}⁻¹(·;x(t) evaluated at x(t+τ).

This section describes the dataset used to train the normalizing flow. First, the MD trajectories are generated by simulating eq 2 using the OpenMM MD library. In this disclosure the focus is on simulating the behavior of small proteins (peptides) in implicit water—i.e., without explicitly modelling the degrees of freedom of the water molecules. Specifically, a dataset of trajectories [symbol] ={ [symbol] }ᵢ₌₁^{P} is generated, where P is the number of distinct peptides considered in the dataset. For each peptide i, a long MD trajectory is generated, which is temporally sampled with a spacing of τ, so that [symbol] =(x(0), x(τ), x(2τ), . . . ). During training, pairs x(t), x(t+τ) are sampled randomly from [symbol] . Each pair x(t), x(t+τ) represents a sample from the conditional distribution u(x(t+c)|x(t)). These samples are then used as examples to train the parameters θ of the flow. Details on the objective function are also provided in the Training Objective subsection hereinabove. Since the flow is trained on trajectory data from a plurality of peptides, it can be deployed at test time to generalize to other peptides absent from the training data. This is in contrast to models such as Boltzmann generators, which are only trained on configurations of a single protein, and cannot generalize to other proteins.

Typically in molecular simulations, the primary interest is in the equilibrium distribution of the positions x^{p}, since the velocities x^{v} in the Boltzmann distribution (eq 1) are normally distributed and independent of the positions, and hence can be sampled trivially. Thus, it is not necessary for x^{v}(t), x^{v}(t+τ) to represent the actual velocities of the atoms in eq 3. Rather, the learning problem is simplified by treating the variables x^{v} as non-physical auxiliary variables within the augmented normalizing flow framework. In detail, for each datapoint x(t)=x^{p}(t), x^{v}(t) in the training dataset [symbol] , instead of obtaining x^{v}(t) by recording the velocities in the MD trajectory, the MD velocity is discarded, and x^{v}(t)˜ [symbol] N (0, I) are drawn independently. Hence at all times t, x^{v}(t) follows a standard normal distribution. The auxiliary variables x^{v} (t) now contain no information about the future state x^{p}(t+τ), x^{v}(t+τ), since x^{v}(t) is drawn independently from every other variable. The transformation in eq 3 can be simplified, therefore, to depend only on x^{p}(t), i.e., x^{p}(t+τ), x^{v}(t+τ):=ƒ_{θ}(z^{p}, z^{v}; x^{p}(t). Likewise, the conditional density of the model can be simplified from p_{θ}(x(t+τ)|x(t)) to p_{θ}(x(t+τ)|x^{p}(t)).

Treating x^{v} as auxiliary variables instead of physical quantities raises the question of why an augmentation is necessary: one could instead attempt to learn a normalizing flow that directly models p_{θ}(x^{p}(t+τ)|p_{θ}(x^{p}(t)), without requiring x^{v}. Auxiliary variables are included for two reasons, however. First, they provide a way to increase the expressivity of the proposal for x^{p} without a prohibitive increase in computational cost [Huang, C., Dinh, L., and Courville, A. C. Augmented normalizing flows: Bridging the gap between generative flows and latent variable models. ArXiv, abs/2002.07101, 2020]. Second, the task of constructing a RealNVP that respects the relevant physical symmetries, in this case permutation equivariance, is simplified with the addition of auxiliary variables. This is discussed in more detail in the Symmetries subsection hereinafter.

This section describes how to use augmented normalizing flow to accelerate sampling from the Boltzmann distribution. Once the flow p_{θ}(x(t+τ)|x(t)) has been trained, it is used as a proposal distribution in a Markov-chain Monte Carlo (MCMC) method to target the joint distribution of the equilibrium positions x^{p} and the auxiliary variables x^{v}, which has density:

$${μ}_{a u g} ( {x}^{p} , {x}^{v} ) = \frac{1}{Z} exp ( - \frac{U ( {x}^{p} )}{{k}_{B} T} ) 𝒩 ( {x}^{v} ; 0 , I ) . \qquad ( 4 )$$

In detail, let m index the states in the Markov chain. Then, starting from an initial state X₀=(X₀^{p}, X₀^{v})∈ [symbol] at m=0, and iterating:

$${\tilde{X}}_{m} ∼ {p}_{θ} ( \cdot \vert {X}_{m}^{p} ) \qquad ( 5 )$$

$${X}_{m + 1} := \{ \begin{matrix}{\tilde{X}}_{m} & with probability α ( {X}_{m} , {\tilde{X}}_{m} ) \\ {X}_{m} & with probability 1 - α ( {X}_{m} , {\tilde{X}}_{m} )\end{matrix} \qquad ( 6 )$$

where α(Xₘ, \tilde{X}ₘ) is the Metropolis-Hastings (MH) acceptance ratio targeting the augmented joint density in eq 4:

$$α ( X , \tilde{X} ) = min \{ 1 , \frac{{μ}_{a u g} ( \tilde{X} ) {p}_{θ} ( X \vert {\tilde{X}}^{p} )}{{μ}_{a u g} ( X ) {p}_{θ} ( \tilde{X} \vert {X}^{p} )} \} \qquad ( 7 )$$

Note that the RealNVP architecture used for p_{θ} allows for efficient sampling and efficient exact likelihood evaluation, which provides fast implementation of eqs 5 and 7.

Additionally, after each MH step, the auxiliary variables X^{v} are resampled using a Gibbs sampling update, which for eq 4 amounts to sampling X^{v} from a standard normal distribution:

$$( {X}_{m}^{p} , {X}_{m}^{v} ) ← ( {X}_{m}^{p} , ϵ ) , ϵ ∼ 𝒩 ( 0 , I ) . \qquad ( 8 )$$

Starting from an initial state, iterating the MH updates (interleaved with the Gibbs sampling updates) yields a sample Xₘ^{p}, Xₘ^{v}˜μ_{aug} (X^{p}, X^{v}) as m→∞. In order to then obtain a sample from the configurational Boltzmann distribution X^{p}˜μ(X^{p}), the auxiliary variables Xₘ^{v} are simply discarded.

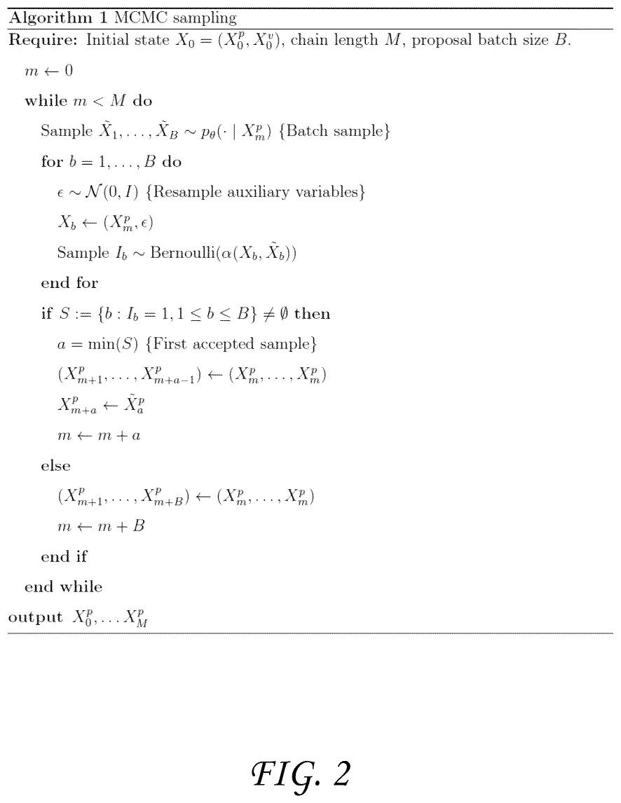

In practice, sending m→∞ is infeasible. Instead, the approach herein is to fix a computational budget and simulate the chain until the budget is reached. The disclosed MCMC scheme is of practical utility if the normalizing flow proposal distribution allows the chain to explore the metastable states of the Boltzmann distribution faster than standard MD simulation, enabling accelerated sampling from μ(X^{p}). Algorithm 1, shown in FIG. 2, provides example pseudocode for the MCMC procedure. Note that computation can be sped up dramatically by sampling a batch of B proposal states from the model at a time.

### Model Architecture

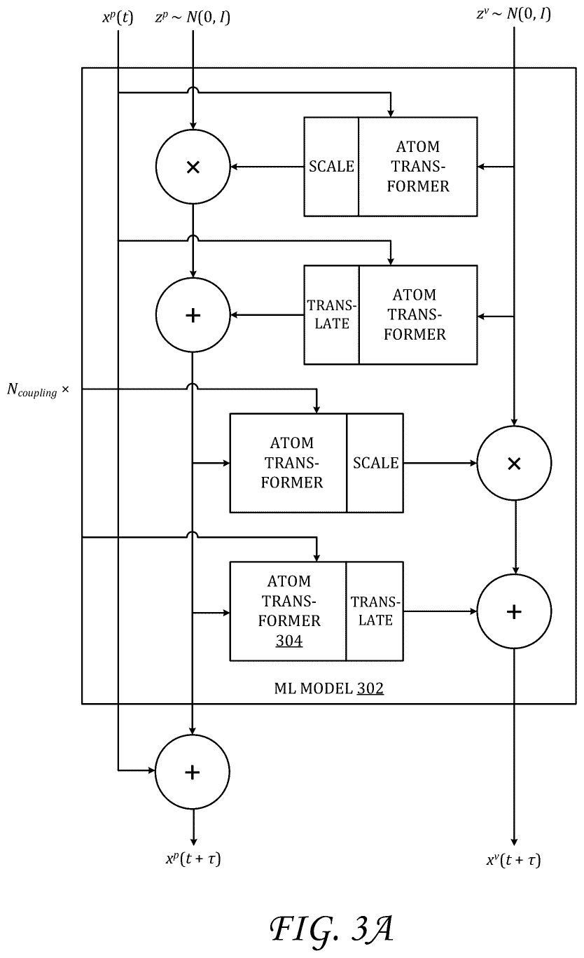

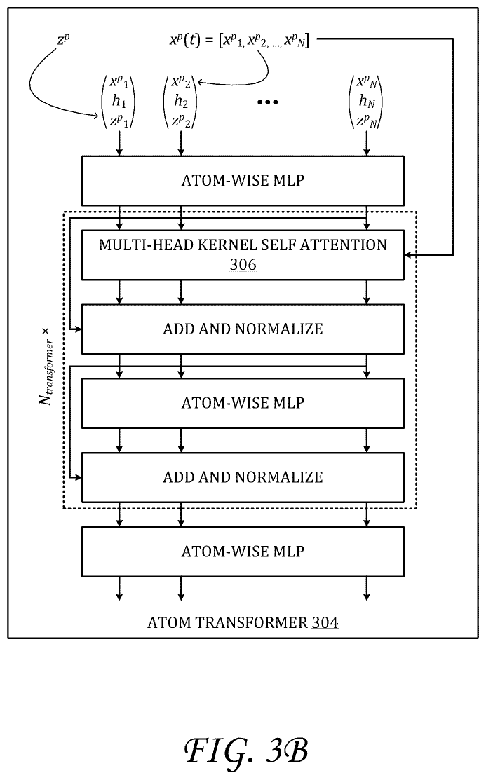

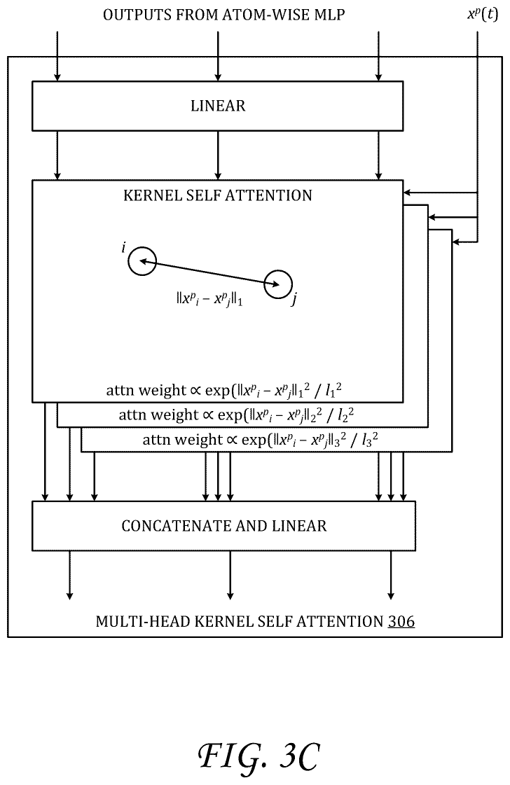

FIGS. 3A through 3C provide a schematic illustration of an example conditional flow architecture of an MD accelerator program. More specifically, FIG. 3A shows a single conditional RealNVP coupling layer of a machine-learning (ML) model 302 in one example. The latent variables z^{p}∈ [symbol] , z^{v} ∈ [symbol] are sampled from a standard normal distribution. They are then passed through N_{coupling} RealNVP coupling layers, where the scale and translation for z^{p} depends on z^{v} and vice versa via atom-transformer modules 304. All transformations are also conditioned on the initial molecular positions, x^{p}(t)∈ [symbol] .

FIG. 3B shows aspects of a single atom-transformer module 304 in one example. The detailed configuration of atom-transformer module 304 differs from the transformer architecture introduced by Vaswani et al. in Attention is All You Need (CoRR, 2017) at least in the following respects: (i) there is no positional embedding as the transformer acts on a set of atoms instead of a sequence of language tokens, (ii) there is an atom-wise multi-layer perceptron (MLP) before and after the N_{transformer} transformer blocks, and (iii) a novel kernel self-attention module 306 is used instead of the standard dot-product attention.

FIG. 3C shows aspects of an example multi-head kernel self-attention module 306. For each of the N_{heads} attention heads, the attention weight between two atoms is determined by the distance between those atoms in the conditioning state, x^{p}(t). Each attention head uses a Gaussian kernel with a different length scale to compute the attention weights.

The architecture of the proposed conditional normalizing flow ƒ_{θ}(z^{p}, z^{v}; x^{p}(t)) will now be described more particularly in the following non-limiting example.

The architecture illustrated in FIGS. 3A through 3C is based on RealNVP, which includes a stack of coupling layers that affinely transform subsets of the dimensions of the latent variable based on the other dimensions. Specifically, the position variables are transformed based on the auxiliary variables, and the auxiliary variables are transformed based on the position variables. In the eth coupling layer of the flow, the following transformations are implemented:

$${z}_{ℓ + 1}^{p} = {s}_{ℓ , θ}^{p} ( {z}_{ℓ}^{v} ; {x}^{p} ( t ) ) ⊙ {z}_{ℓ}^{p} + {t}_{ℓ , θ}^{p} ( {z}_{ℓ}^{v} ; {x}^{p} ( t ) ) , \qquad ( 9 )$$

$${z}_{ℓ + 1}^{v} = {s}_{ℓ , θ}^{v} ( {z}_{ℓ + 1}^{p} ; {x}^{p} ( t ) ) ⊙ {z}_{ℓ}^{v} + {t}_{ℓ , θ}^{v} ( {z}_{ℓ + 1}^{p} ; {x}^{p} ( t ) ) . \qquad ( 10 )$$

Going forward, the coupling layer index [symbol] is suppressed to reduce clutter. Here ⊙ is the element-wise product, and s_{θ}^{p}: [symbol] → [symbol] is the proposed atom transformer, a neural network based on the transformer architecture that takes the auxiliary latent variables z^{v} and the conditioning state x(t) as input and uses them to output scaling factors for the position latent variables z^{p}. The function t_{θ}^{p}: [symbol] → [symbol] is implemented as another atom transformer, which uses z^{v} and x(t) to output a translation of the position latent variables z^{p}.

The affine transformations of the position variables, in eq 9, are interleaved with similar affine transformations for the auxiliary variables, in eq 10. Since the scale and translation factors for the positions depend only on the auxiliary variables, and vice versa, the Jacobian of the transformation is lower triangular, allowing for efficient computation of the density. Each application of eq 9 followed by eq 10 represents a single coupling layer of the flow. The full flow ƒ_{θ} consists of N_{coupling} stacked coupling layers, beginning from z˜ [symbol] (0, I) and ending with a sample from p_{θ}(x(t+τ)|x(t)). This is depicted in FIG. 3A. Note that there is a skip connection such that the output of the coupling layers predicts the change x(t+τ)−x(t), rather than outputting x(t+τ) directly.

The architecture of the proposed atom transformer network which is used to output the scale and translation factors in each coupling layer will now be described. The transformer architecture allows the model to be easily applied to molecules with varying numbers of atoms. The atom transformer works by first converting the N atoms in the molecule into a sequence of N vectors for the transformer to act on. Specifically, let xᵢ^{p}(t), zᵢ^{p}, zᵢ^{p}, all elements of [symbol] , denote respectively the position of atom i in the conditioning state, the position latent variable for atom i, and the auxiliary latent variable for atom i. To implement an atom transformer which takes z^{v} as input, such as s_{θ}^{p} (z^{v}, x(t) and t_{θ}^{p}(z^{v}, x(t)) in eq 9, the variables associated with atom i are first concatenated. This leads to a vector αᵢ: =[xᵢ^{p}(t), hᵢ, zᵢ^{v}]^{T} ∈ [symbol] , where zᵢ^{p} has been excluded since s_{θ}^{p}, t_{θ}^{p} are not allowed to depend on z^{p}. Here hᵢ∈ [symbol] is a learned embedding vector which depends only on the atom type—e.g., carbon, oxygen, nitrogen, etc. The vector αᵢ is then fed into an MLP ϕᵢₙ: [symbol] → [symbol] , where D is the feature dimension of the transformer. This same MLP acts atom-wise on each vector. The sequence of N MLP output vectors ϕ(α₁), . . . , ϕ(α_{N}), each of dimension D, is then fed into N_{transformer} stacked transformer layers. After the transformer layers, the N output vectors of dimension D are then passed through another atom-wise MLP, ϕₒᵤₜ: [symbol] → [symbol] . Hence the final output is in [symbol] , which is the appropriate shape for the scale and translation factors. This is depicted in FIG. 3B. A similar procedure is performed on the vector [xᵢ^{p}(t), hᵢ, zᵢ^{p}]^{T}, now including zᵢ^{p} but excluding zᵢ^{v}, when implementing s_{θ}^{v} and t_{θ}^{v} from eq 10.

Two differences in the transformer layers used in the subject implementation as compared to a conventional transformer architecture are as follows. First, in order to maintain permutation equivariance, a positional encoding is not used in the subject implementation. Second, in the subject implementation instead of the standard multi-head dot product attention, a simple multi-head kernel self-attention module is introduced, which is described next.

The kernel self-attention module is motivated by the observation that the force field interactions between the atoms in the molecule are local—i.e., they act more strongly on atoms that are nearby than atoms that are farther way. Intuitively, for values of t that are not too large, the position of the atoms at time t+τ will be more influenced by atoms that are nearby at time t, compared to atoms that are far away at time t. To incorporate this geometric inductive bias into the architecture, the dot product attention is replaced by the proposed kernel self-attention. In kernel self attention, the weight wᵢⱼ for atom i attending to atom j is given by:

$${w}_{i j} = \frac{exp ( - {‖ {x}_{i}^{p} - {x}_{j}^{p} ‖}_{2}^{2} / {ℓ}^{2} )}{{\sum }_{j = 1}^{N} exp ( - {‖ {x}_{i}^{p} - {x}_{j}^{p} ‖}_{2}^{2} / {ℓ}^{2} )} . \qquad ( 11 )$$

The output vectors {r_{out,i}}ᵢ₌₁^{N} of kernel self attention, given the input vectors {r_{in,i}}ᵢ₌₁^{N}, are then computed as:

$${r}_{o u t , i} = {\sum }_{j = 1}^{N} {w}_{i j} V {r}_{in , j} , \qquad ( 12 )$$

where V is a learned linear layer. In other words, the attention weight is given by a Gaussian kernel function of the Euclidean distance between the two atoms in the conditioning state. The Gaussian kernel has a length scale [symbol] , which can either be set as a hyperparameter or learned during training. A multi-head version of kernel self attention is introduced also, where each head attends with attention weights given by eq 11, but with each head using its own length scale. Hence there is one length scale parameter for each head, [symbol] ₁, . . . , [symbol] _{N}_{_{heads}}. The kernel self attention module is illustrated in FIG. 3C. In experiments it is found that kernel self attention was significantly faster to compute than dot product attention, and produced similar or improved performance.

### Training Objective

The objective function used to train the MD accelerator will be described next. The model is trained in two stages. In the first stage, likelihood training, the model is trained via maximum likelihood on pairs of states from the trajectories in the dataset. In the second stage, acceptance training, the model is fine-tuned to maximize the probability of acceptance in eq 5.

Let k index training pairs, such that {x^{(k)}(t), x^{(k)}(t+τ)}ₖ₌₁^{K} represents all pairs of states at times t apart in the dataset [symbol] from Section 3.2. During likelihood training, the conditional likelihood of the future state is optimized with respect to θ:

$${ℒ}_{lik} ( θ ) = \frac{1}{K} {\sum }_{i = 1}^{K} log {p}_{θ} ( {x}^{( k )} ( t + τ ) \vert {x}^{( k )} ( t ) ) . \qquad ( 13 )$$

Once likelihood training is complete, it is found that in some cases the MH acceptance probabilities are too low to allow efficient sampling from the Boltzmann distribution. To address that issue, a fine-tuning stage is added in order to optimize the acceptance probability. Let x^{(k)}(t) be sampled uniformly from [symbol] . Then, the model is used to sample \tilde{x}_{θ}^{(k)}(t+τ)˜p_{θ}(·|x^{(k)}(t)) using eq 3. Note that the sample value depends on θ through ƒ_{θ}. This is used to optimize the acceptance probability in eq 7 with respect to θ. Let re (X, \tilde{X}) denote the model-dependent term in the acceptance ratio in eq 7:

$${r}_{θ} ( X , \tilde{X} ) := \frac{{μ}_{a u g} ( \tilde{X} ) {p}_{θ} ( X \vert {\tilde{X}}^{p} )}{{μ}_{a u g} ( X ) {p}_{θ} ( \tilde{X} \vert {X}^{p} )} \qquad ( 14 )$$

Then the acceptance objective is given by:

$${ℒ}_{acc} ( θ ) = \frac{1}{K} {\sum }_{k = 1}^{K} log {r}_{θ} ( {x}^{( k )} ( t ) , {\tilde{x}}_{θ}^{( k )} ( t + τ ) ) \qquad ( 15 )$$

In practice it is found that training to maximize the acceptance probability can lead to the model proposing changes that are too small: if \tilde{x}_{θ}^{(k)}(t+τ)=x^{(k)}(t), then all proposals will be accepted. To mitigate this, during acceptance training, the model is trained on an objective that consists of a weighted average of [symbol] _{acc}(θ), [symbol] ₗᵢₖ(θ) and a Monte Carlo estimate of the average differential entropy,

$${ℒ}_{ent} ( θ ) := - \frac{1}{K} {\sum }_{k = 1}^{K} log {p}_{θ} ( {\tilde{x}}_{θ}^{k} ( t + τ ) \vert {x}^{( k )} ( t ) ) . \qquad ( 16 )$$

The weighting factors for each of the three terms are hyperparameters of the training procedure.

### Symmetries

The Langevin dynamics simulated in eq 2 respects certain physical symmetries that would be advantageous to incorporate into the model. These are (i) permutation equivariance, (ii) translation equivariance and (iii) rotation equivariance. How each of these symmetries is incorporated in the MD accelerator will now be described.

Let σ be a member of the permutation group on N elements, σ∈ Perm(N). Consider the state of the molecule, x=(x^{p}, x^{v})∈ [symbol] . The action of σ on x is defined simply as permuting the ordering of the atoms in the state:

$$\begin{matrix}σ x = σ ( {x}^{p} , {x}^{v} ) & ( 17 ) \\ = σ ( ( {x}_{1}^{p} , … , {x}_{N}^{p} ) , ( {x}_{1}^{v} , … , {x}_{N}^{v} ) ) & ( 18 ) \\ = ( ( {x}_{σ ( 1 )}^{p} , … , {x}_{σ ( N )}^{p} ) , ( {x}_{σ ( 1 )}^{v} , … , {x}_{σ ( N )}^{v} ) ) & ( 19 )\end{matrix}$$

Since the atoms in a molecule have no intrinsic ordering, the physical evolution of the system is unaffected by a permutation of the ordering of the atoms in the vector x. Hence, if such a permutation is performed on the state x(t), the only effect on the future state x(t+τ) is to permute the atoms similarly—i.e.,

$$μ ( σ x ( t + τ \vert σ x ( t ) ) = μ ( x ( t + τ ) \vert x ( t ) ) . \qquad ( 20 )$$

The MD accelerator architecture described in the Model Architecture subsection hereinabove satisfies permutation equivariance exactly. To show this, the following proposition is used, which extends conventional approaches for conditional flows, as proved in the Proof of Proposition subsection hereinafter:

Proposition. Let σ be a symmetry action, and let ƒ(·;·) be an equivariant map such that ƒ(σz; σx)=σƒ(z; x) for all σ, z, x. Further, let the base distribution p(z) satisfy p(σz)=p(z) for all σ, z. Then the conditional flow defined by z˜p(z), x(t+τ):=ƒ (z; x(t)) satisfies p(σx(t+τ)|σx(t)=p(x(t+τ)|x(t).

The function ƒ_{θ}(z; x(t)) in eq 3 is jointly permutation-equivariant with respect to both the latent variable z and the conditioning state x(t):

$${f}_{θ} ( σ z ; σ x ( t ) ) = σ {f}_{θ} ( z ; x ( t ) ) . \qquad ( 21 )$$

This is because the transformer architecture is permutation-equivariant without the positional encodings, and permuting z and x(t) with the same permutation σ permutes the vector inputs to the transformer, as shown in FIG. 3B. Furthermore, the base distribution p(z)= [symbol] (0, I) is permutation invariant. Hence, by the proposition above, the MD accelerator conditional flow model is permutation-equivariant. It will be noted that the presence of auxiliary variables allows construction of a permutation-equivariant coupling layer. If the flow only took z^{p} as input without z^{v}, then each coupling layer would have to unnaturally split the Cartesian components of z^{p} into two disjoint sets, and affinely transform some coordinates based on the others. The presence of z^{v} allows all the components of z^{p} to be transformed together.

Consider a Euclidean transformation T=(R, α) that acts on the positions x^{p} as follows:

$${Tx}_{i}^{p} = {Rx}_{i}^{p} + a , 1 \leq i \leq N \qquad ( 22 )$$

where R is a 3×3 rotation matrix, and α∈ [symbol] is a translation vector. The model should also satisfy p_{θ}(Tx(t+τ)|Tx(t))=p_{θ}(x(t+τ)|x(t)), where T acts only on the positions and not the auxiliary variables. Translation equivariance is achieved by canonicalization—i.e., subtracting the average position of the atoms in the initial molecular state from both the initial and target positions. More details are provided in the Translation Invariance via Canonicalization subsection hereinafter. Rotation equivariance, on the other hand, is not incorporated into the architecture but is instead handled by data augmentation: each training pair (x(t), x(t+τ)) from the dataset D is acted upon by a random rotation matrix R to form (Rx(t), Rx(t+τ)) before using it to train the model, producing a training data set with pairs that exhibit rotational variations.

Deep-learning techniques have been applied to molecular modeling, some approaches implementing Boltzmann generators. A Boltzmann generator relies on a normalizing flow architecture to sample from the Boltzmann distribution. Significantly, the Boltzmann generator uses internal coordinates to parameterize a given protein, which is a barrier to generalizing across multiple proteins, unlike the MD accelerator herein.

Diffusion models are another class of deep-generative models that have been applied to molecular modeling. Recently, the conditional-diffusion model GeoDiff has been proposed, which predicts molecular conformations (i.e., position variables) given a molecular graph (i.e., atom and bond types). Unlike the MD accelerator herein, GeoDiff was not applied to proteins, but instead to small molecules. Furthermore, since GeoDiff does not have any form of Metropolis-Hastings correction, there is no way to guarantee that it provides unbiased samples from the Boltzmann distribution. Very recently, other models, referred to as Chroma and RoseTTAFold diffusion, have been introduced as diffusion models for de novo protein design. Unlike the MD accelerator of the present disclosure, the focus of these models is to design novel protein structures and sequences, rather than to accurately simulate the thermodynamic properties of a given protein. In particular, they are unable to sample the various metastable states of a protein from the Boltzmann distribution.

AlphaFold2 is another deep-learning model that predicts the 3-dimensional, folded structure of a protein from its amino acid sequence and multiple-sequence alignment (MSA) data. AlphaFold2 relies on a variant of the transformer architecture. However, unlike the atom transformers of the MD accelerator herein, the Evoformer transformer blocks of the AlphaFold2 model are rotationally and translationally equivariant. Since AlphaFold2 only predicts a single folded state from a given amino acid sequence, it does not provide information about the relative probability of various metastable states in the Boltzmann distribution. Furthermore, its architecture relies on the representation of proteins as a sequence of amino acids, and cannot be modified without significant further development to handle other molecules. In contrast, since the MD accelerator of the this disclosure uses an all-atom representation in Cartesian coordinates, it can be applied to non-protein molecules.

Hamiltonian Monte Carlo (HMC) employs auxiliary variables but relies on Hamiltonian dynamics instead of a learned proposal distribution. A-NICE-MC [Song, J., Zhao, S., and Ermon, S. A-nice-mc: Adversarial training for MCMC. Advances in Neural Information Processing Systems, 30, 2017.] uses a volume-preserving normalizing flow as a Markov chain kernel, but that flow is trained with a likelihood-free adversarial method.

Continuing, now, with the MD-accelerator description, three series of computer-simulation experiments were performed on small peptide systems of different sizes to show the sampling properties as well as the transferability of the model. In comparing model-generated trajectories with conventional MD, the slowest transitions between metastable states are of greatest interest. To find the transitions as well as the metastable states, a time-lagged independent component analysis (TICA) is used. Moreover, results from the present model and conventional MD are compared in terms of effective sample-size-per-second wall-clock time (ESS/s) for these transitions. The slowest transition, which is along the first TICA component (TIC 0), is of particular interest as it takes the longest to decorrelate. The ESS/s is given by

$$ESS / s = \frac{{N}_{eff}}{{t}_{sampling}} = \frac{N}{{t}_{sampling} ( 1 + 2 {\sum }_{τ = 1}^{∞} {ρ}_{τ} )} , \qquad ( 23 )$$

where Neff is the effective number of total samples, tsampling is the sampling wall-clock-time in seconds, and pr is the autocorrelation for the lag time t. The training for all models starts with the likelihood objective eq 13 and is continued with a combination of eq 13, eq 15, and eq 16.

All datasets are created with MD simulations performed with the same parameters, as listed in the Data-set Details subsection hereinafter. The three investigated datasets are (i) alanine dipeptide (AD), (ii) dipeptides consisting of two amino acids (2AA), and (iii) tetrapeptides consisting of 4 amino acids (4AA). Table 1 provides additional details. The relative frequencies of the amino acids are similar across the different splits of the datasets. Note that for the 4AA dataset, the training set consists of only about 1% of the total number of tetrapeptides.

TABLE 1
Dataset details.

| | | | |
|---|---|---|---|
| Dataset name | AD | 2AA | 4AA |

| | | | | | | |
|---|---|---|---|---|---|---|
| Training set simulation time | 100 | ns | 50 | ns | 50 | ns |
| Test set simulation time | 100 | ns | 1 | μs | 1 | μs |
| MD integration step Δt | 0.5 | fs | 0.5 | fs | 0.5 | fs |
| Lag time τ | 0.5 | ns | 0.5 | ns | 0.05 | ns |

| | | | |
|---|---|---|---|
| # training peptides | 1 | 200 | 1400 |
| # training pairs per peptide | 2E+05 | 1E+04 | 1E+04 |
| # test peptides | 1 | 100 | 30 |

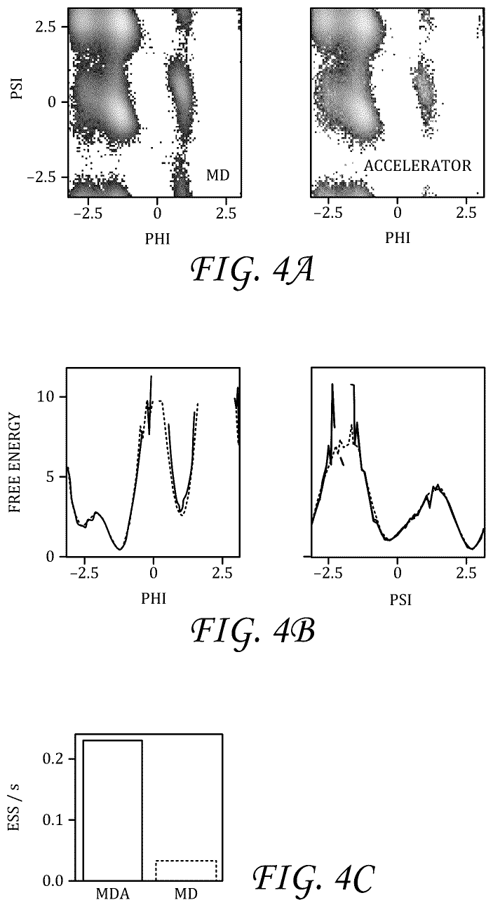

Alanine dipeptide was investigated as a small single peptide example. A Markov chain for alanine dipeptide is generated with the trained MD accelerator according to Algorithm 1, accepting about 2% of the proposed states. FIGS. 4A through 4C show example results of alanine dipeptide experiments. In particular FIG. 4A shows Ramachandran plots for MD and model samples; FIG. 4B shows a free-energy comparison between MD (dashed line) and the MD accelerator (solid line), along the two dihedral angles φ and ψ; and FIG. 4C shows the speed-up in EES/s of the MD accelerator model relative to MD. As shown in FIGS. 4A and 4B, all metastable states are recovered with the correct relative weights. Moreover, the autocorrelation along the σ dihedral angle decays much faster in terms of wall-clock-time, resulting in ˜7× speed-up relative to MD. This is partly due to the model proposing rare transitions much more frequently.

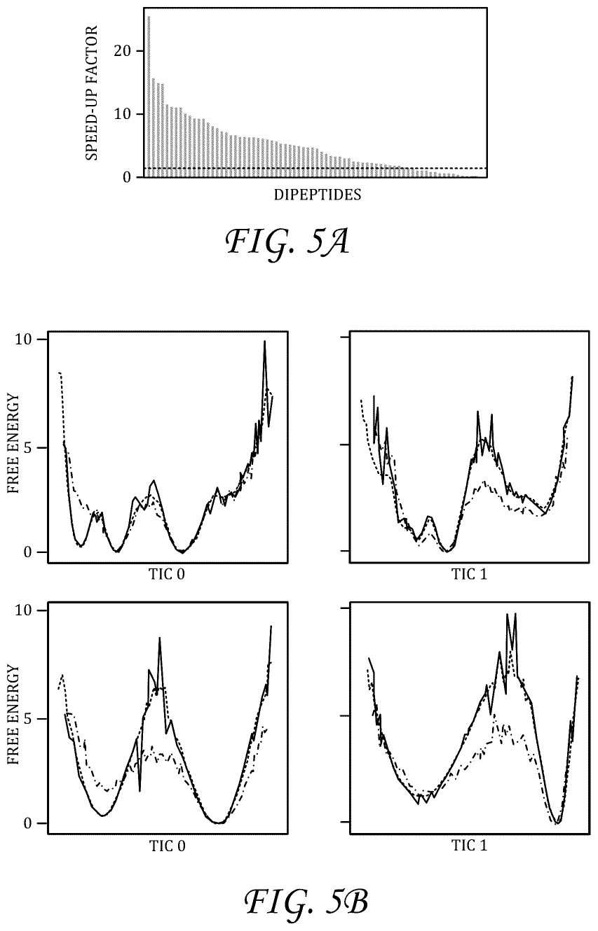

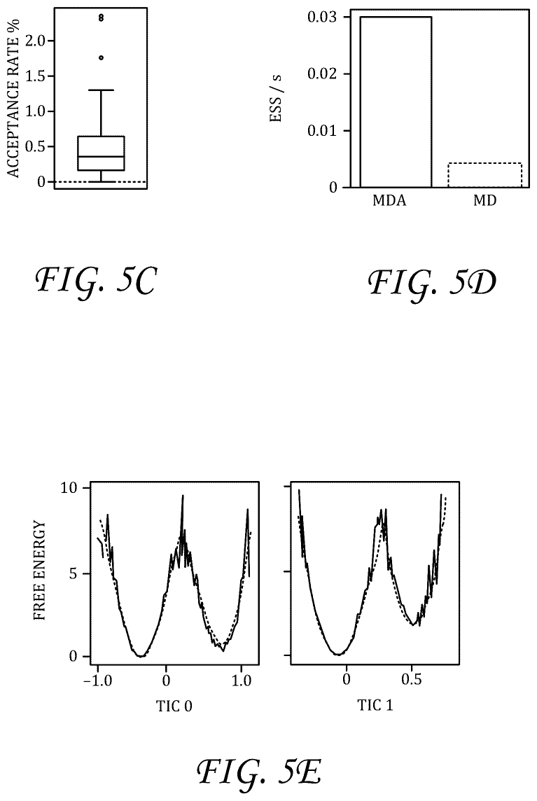

FIGS. 5A through 5E show example results of additional dipeptide experiments. In particular, FIG. 5A shows the speed-up in ESS/s for the slowest TICA component from the MD-accelerator (solid line) relative to MD (dashed line). The horizontal dashed line is drawn at a relative speed-up of one. FIG. 5B shows TICA plots for the dipeptide histidine-proline (HP) from the MD-accelerator in combination under MCMC (solid line) and in exploration mode (dot-dashed line), both relative to MD (dashed line). FIG. 5C shows acceptance probabilities for the validation set. The horizontal dashed line in FIG. 5C is drawn at the 0.01% acceptance rate, below which sampling is difficult. FIG. 5D shows speed-up in EES/s for the MD accelerator relative to MD for histidine-glutamine (HQ), and FIG. 5E is a free-energy comparison along TIC 0 and TIC 1 for HQ.

The second series of experiments demonstrates the transferability of the method to generic dipeptides. Again, the trained MD accelerator samples according to Algorithm 1. The model is capable of sampling nearly all of the dipeptides efficiently—i.e., the acceptance probability is greater than 0.01% for all peptides (FIG. 5C), and all metastable states are explored. Only for three of the 80 unseen dipeptides does the method fail to explore all metastable states within 20 million steps. These are excluded from the speed-up calculations. Moreover, the model has a median speed-up factor of about five compared to MD in terms of ESS/s. As an example, results for the dipeptide HQ are shown, which has the median speed-up factor. Here all metastable states are found with the correct relative weights (FIGS. 5B and 5E), while the autocorrelation for the slowest TICA component decays faster than for MD, resulting in ˜5× wall-clock speed-up, as shown in FIG. 5D.

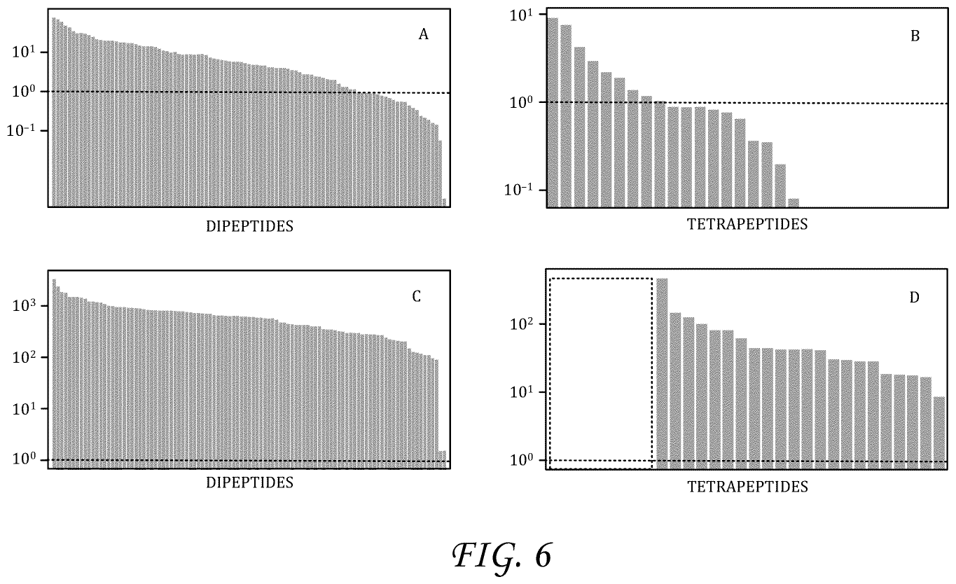

FIG. 6 shows speed-up factors in terms of ESS/s ratios for the slowest TICA component of the MD accelerator under MCMC and exploration algorithms, relative to MD. In all four panels the horizontal dashed red line shows a speed-up factor of one. The area to the right of panels B and D depict peptides for which the MD accelerator fails to explore all metastable states within 20 million steps, but MD does. The dashed area in panel D encloses peptides where MD fails to find all metastable states, but the MD accelerator does. More particularly, panels A and B show the speed-up for the MD-accelerator MCMC algorithm (Algorithm 1) on test dipeptides (2AA) and tetrapeptides (4AA), respectively. Panels C and D show the speed-up for the MD-accelerator exploration algorithm (Algorithm 2) on test dipeptides (2AA) and tetrapeptides (4AA), respectively.

Panel A shows the speed-up achieved by the MD accelerator, relative to MD, for each of the 100 test dipeptides. The MD accelerator provides a median speed-up factor of about five across these dipeptides. In addition, the MD accelerator was used to generate samples with without the MH correction, as detailed hereinafter, in the context of Algorithm 2. Panel C shows the result of sampling 100 parallel chains at only 10000 steps starting from the same initial state for each dipeptide in the test set, where a single chain is selected in order to finds all metastable states for evaluation. As before, the ESS/s is computed for comparison against MD. These results show a median speedup factor of ≈600. It will be noted that the actual speedup when using all the chains sampled in parallel would be much larger.

MD-accelerator exploration leads to free-energy estimates that are qualitatively similar to those of MD, but less accurate than the MD-accelerator/MCMC implementation (as shown in FIG. 5B).

Results of the more challenging tetrapeptide (4AA data set) study are also presented in FIG. 6. After training on the training set, 20 million Markov chain states were sampled for each test tetrapeptide using Algorithm 1 and compared with long MD trajectories (1 μs=2×109 timesteps). In contrast to the simpler dipeptides, both the MD-accelerator/MCMC implementation and the long MD trajectories miss some metastable states. However, the MD accelerator in exploration mode (Algorithm 2) can be used as a validation tool to quickly verify exploration of the whole state space.

Although the MD accelerator failed to explore all metastable states with Metropolis-Hastings for some peptides, the accelerator still may be used to explore all metastable states efficiently. To that end the MD accelerator, trained on the tetrapeptides, is combined with MD for fast exploration of metastable states of previously unseen peptides. The exploration algorithm is described in the Exploration of Metastable States subsection hereinafter. The algorithm takes about 5 minutes (wall-clock time) for tetrapeptides, which is orders of magnitude faster than running MD instead. However, this comes at the cost of no longer sampling from the Boltzmann distribution.

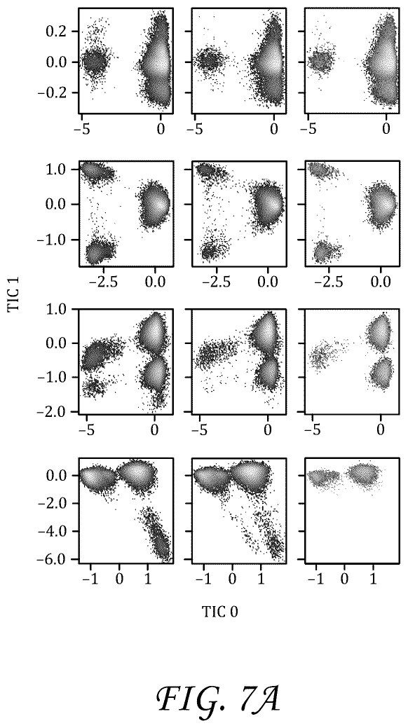

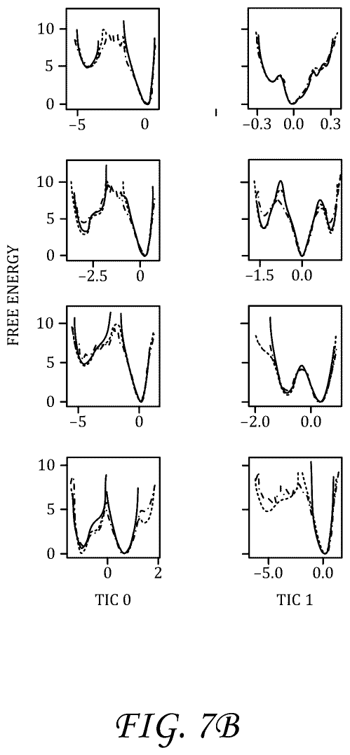

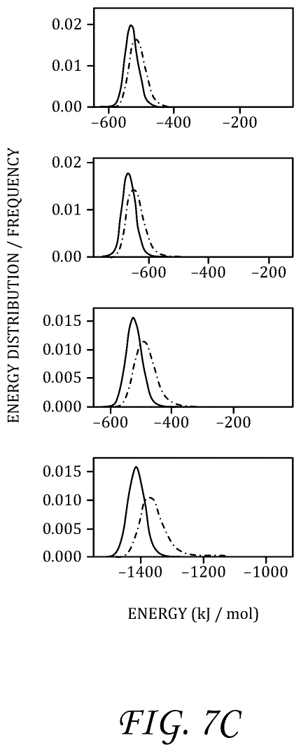

FIGS. 7A through 7C show example results of additional experiments on tetrapeptides-viz., results of four examples from the validation set comparing MD (referring to pure MD simulation), MCMC (samples generated with the MD accelerator model according to Algorithm 1), and a fast exploration algorithm (samples generated with the MD accelerator model according to Algorithm 2). FIG. 7A shows TICA plots for the trajectories. FIG. 7B shows projections on the first two TICA components, and FIG. 7C shows potential energies. Again, the solid line represents MD-accelerator results under MCMC, the dot-dashed line represents MD-accelerator results in exploration mode, and the dashed line represents the comparative MD computation. Each row corresponds to a different tetrapeptide from the validation set, serine-alanine-glutamine-alanine (SAGA), aspartic acid-proline-alanine-serine (DPAS), leucine-alanine-lysine-serine (LAKS), and methionine-glycine-arginine-serine (MGRS), respectively. Not all metastable states are explored with the MCMC algorithm for the latter two, but are found using the exploration algorithm. In all cases all metastable states are explored with the exploration algorithm, as shown in FIG. 7A, yielding samples at slightly too-high energies, as reported in FIG. 7C. For the first two tetrapeptides, SAGA and DPAS, all metastable states are explored with the MCMC algorithm, and the relative weights are correctly recovered, as shown in FIG. 7B. For the later two tetrapeptides, LAKS and MGRS, the MCMC algorithm misses one transition.

As described herein, this disclosure presents an MD accelerator, a novel conditional normalizing-flow architecture and MCMC procedure to sample from the Boltzmann distribution of molecules. It is shown in computer-simulated experiments on small peptides that the MD accelerator of the present disclosure accelerates wall-clock sampling time on peptides unseen in the training distribution. This work represents a technical advance towards accurate and transferable deep learning systems for enhanced molecular sampling.

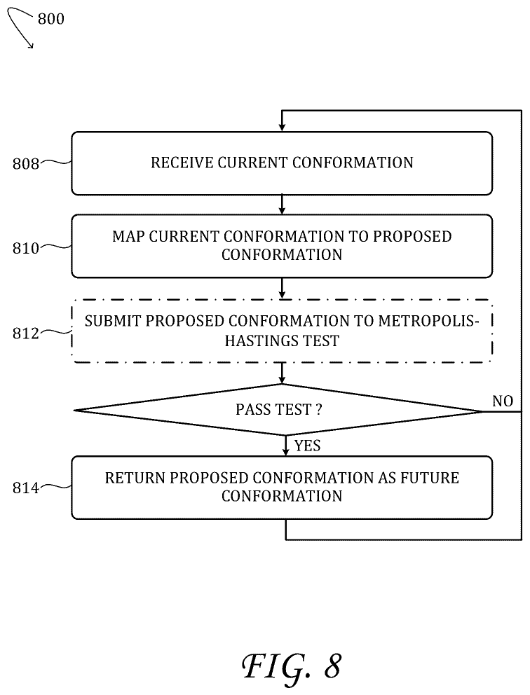

FIG. 8 shows aspects of an example method 800 to forecast a future conformation of a molecule based on a current conformation of the molecule. In some examples the molecule comprises an oligopeptide, polypeptide, protein, biomolecule, and/or polymer. In method 800 at least the future conformation is a sample drawn from a Boltzmann distribution of conformations of the molecule. In some examples both the current and future conformations are samples drawn from the Boltzmann distribution. In some examples the future conformation corresponds to a metastable state of the molecule. Generally speaking, each of the conformations received comprise nuclear coordinates on a Cartesian coordinate system or on a coordinate system obtained from the Cartesian coordinate system by a linear transformation.

At 808 of method 800, the current conformation is received in a machine-learning model that has been previously trained to map a plurality of conformations received to a corresponding plurality of conformations proposed. In some examples the machine-learning model includes one or more transformer blocks adapted to transform vectorized representations of atomic nuclei corresponding to each of the conformations received. The term ‘transformer block’ should be interpreted in the context of the field of artificial intelligence. Furthermore, each of the one or more transformer blocks may include a multi-head self-attention mechanism. In more particular examples, the multi-head self-attention mechanism may be configured to compute a kernel-weighted self attention, where the influence of a first atomic nucleus on the transformation of a second atomic nucleus varies as a function of distance between the first and second atomic nuclei. In alternative examples the machine-learning model may comprise another type of predictive model, such as a diffusion model.

At 810 the current conformation is mapped to a proposed conformation via the machine-learning model. Typically the proposed conformation is appended to a Markov chain. In examples based on a RealNVP approach, the machine-learning model maps the conformations received to the conformations proposed by enacting an invertible, normalizing-flow function on each sample drawn from a normal distribution around each conformation received. Here the normalizing-flow function is parameterized by learned parameter values of the machine-learning model. In some examples the Markov chain is grown according to a Markov-chain Monte Carlo (MCMC) algorithm.

At 812 the proposed conformation may be submitted to a Metropolis-Hastings test configured to accumulate the Boltzmann distribution without asymptotic bias, by accepting some of the conformations proposed and rejecting the balance of the conformations proposed. In some examples, rejecting the balance of the conformations proposed comprises pruning the balance of the conformations proposed from the Markov chain. At 814 the proposed conformation is returned as the future conformation provided that the proposed conformation is accepted by the Metropolis-Hastings test.

In other examples, method 800 may be performed without submission of the proposed conformation to the Metropolis-Hastings test. In some examples an algorithm other than Metropolis Hastings may be used to preclude asymptotic bias in the generated samples of proposed conformations. In other examples, the sample proposed conformation may simply be output without ensuring that it is not asymptotically biased, for implementations in which freedom from such bias is not required. Such implementations may include the ‘exploration’ methods detailed herein. As an example method of the latter implementation, the method may include receiving the current conformation in a trained machine-learning model that has been previously trained to map a plurality of conformations received to a corresponding plurality of conformations proposed, mapping the current conformation to a proposed conformation via the trained machine-learning model, wherein the proposed conformation is appended to a Markov chain, and returning the proposed conformation as the future conformation.

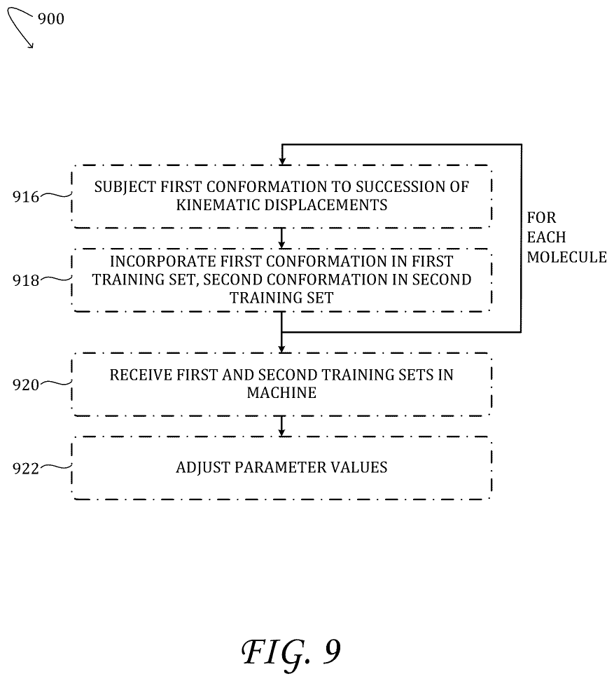

FIG. 9 shows aspects of an example method 900 to train the machine-learning model used in method 800. Accordingly, methods 800 and 900 may be used together in some examples and scenarios; in other examples and scenarios the methods may be used separately. At 916, in particular, for each of a plurality of molecules, a first conformation of that molecule is subjected to a succession of kinematic nuclear displacements to yield a second conformation of the molecule advanced in time by a predetermined interval. Each displacement is based on a temperature of the Boltzmann distribution according to a molecular-dynamics algorithm. In more particular examples the molecular-dynamics algorithm may enact Langevin dynamics. This or any suitable molecular-dynamics simulation may be run on an openMM server, for instance. In some examples the molecular-dynamics algorithm approximates integration of differential equations of motion of the molecule over time using a discretizing timestep. Here the predetermined interval may be at least six orders of magnitude longer than the discretizing timestep. In some examples the predetermined interval is at least one nanosecond.

At 918 the first conformation is incorporated into a first training set, and the second conformation is coordinately incorporated into a second training set. Steps 916 and 918 are repeated for a large number of molecules—optionally, molecules of the same class as the molecules whose conformations are to be sampled. At 920 the first and second training sets are received in the machine-learning model. At 922 parameter values of the machine-learning model are adjusted so as to minimize a residual for mapping elements of the first training set to corresponding elements of the second training set.

Method 800 of FIG. 8 may be used as a stand-alone method or may be incorporated into various container methods. For example, a method to statistically sample the Boltzmann distribution may comprise the step of applying method 800 repeatedly, starting with the same current conformation. In this example each future conformation comprises a sample of the Boltzmann distribution. In another example, a method to estimate a rate constant of isomerization of the current conformation of a molecule to an isomeric conformation may comprise applying method 800 repeatedly, starting with the same current conformation. Then the rate constant is estimated based on a probability that the future conformation approaches the isomeric configuration within the predetermined time increment.

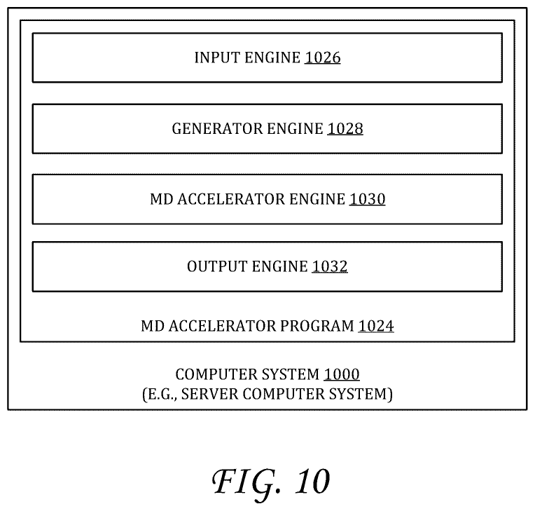

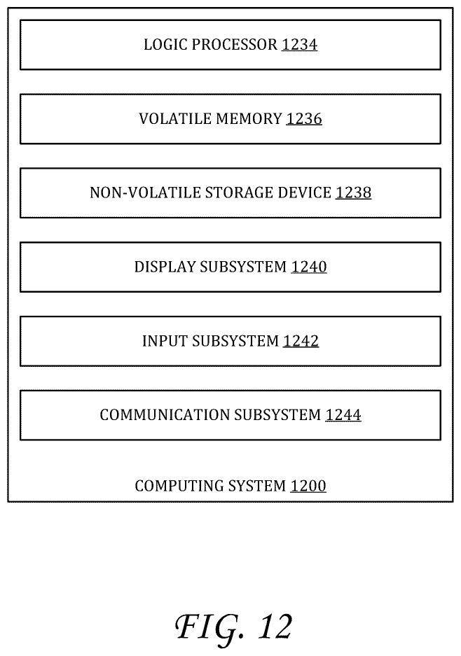

Method 800 may be enacted on a computer system 1000 as shown in FIG. 10, further aspects of which are described with reference to FIG. 12. In one example, the computer system 1000 may be a server computer system configured to communicate over a computer network such as the internet with remote client-computing devices. The computer system 1000 executes an MD accelerator program 1024 to implement software engines 1026 through 1032, which are software engines included within MD accelerator program 1024 and executed on hardware of the computer system. The computing system 1000 implements input engine 1026 of the MD accelerator program 1024, which is configured to receive a primary structure of the molecule. The computer system 1000 further implements a generator engine 1028 of the MD accelerator program 1024, which is configured to generate the current conformation of the molecule based on the primary structure received. The computer system further implements an MD-accelerator engine 1030 of the MD accelerator program 1024, which is configured to execute method 800 on the current conformation. The computer system further implements an output engine 1032 of the MD accelerator program 1024, which is configured to output the future conformation of the molecule as returned by MD-accelerator engine 1030. Computer system 1000 is illustrated in abbreviated form. A more complete description of a suitable computer system that may serve as computer system 1000 is given with reference to FIG. 12, discussed below. Of course, it will be appreciated FIG. 10 is merely one example implementation, and that MD accelerator program 1024 may take a variety of alternative forms and processing strategies to perform the functions described herein. Thus aspects of these functions may be performed via distributed processing strategies or implemented entirely in hardware or firmware.

No aspect of the foregoing drawings or description should be interpreted in a limiting sense, because numerous variations, extensions, and omissions are equally envisaged. For instance, while the methods herein are described as being applied to ‘molecules’, they also may be applied to ensembles of molecules, or, to an unconstrained portion of a molecule wherein another portion has constrained degrees of freedom (e.g., is bound to a host). Hence, the term ‘molecular system’ may be used as a substitute for ‘molecule’ throughout this disclosure. In some examples, accordingly, instead of modelling every atom of a molecular system individually, a coarse-grained representation may be used, where only a subset of the atoms (viz., atoms of particular interest) are modeled, the rest of the molecular system frozen in a low-energy conformation or otherwise abstracted. In a large protein, for instance, the MD-accelerator may be dimensioned to include only the C-alpha atoms. This approach has the potential to significantly reduce computational cost by avoiding unnecessary computation.

### Data set Details

The MD accelerator of the present disclosure was evaluated on different data sets. All data sets are simulated in an implicit solvent using the openMM library. For all MD simulations herein, the parameters from Table 2 are used unless otherwise noted.

TABLE 2
MD simulation details.

| Force Field | amber-14 | |
|---|---|---|
| Time step | 0.5 | fs |
| Friction coefficient | 0.3 | ps⁻¹ |
| Temperature | 310 | K |

| | |
|---|---|
| Integrator | LangevinMiddleIntegrator |

### Exploration of Metastable States

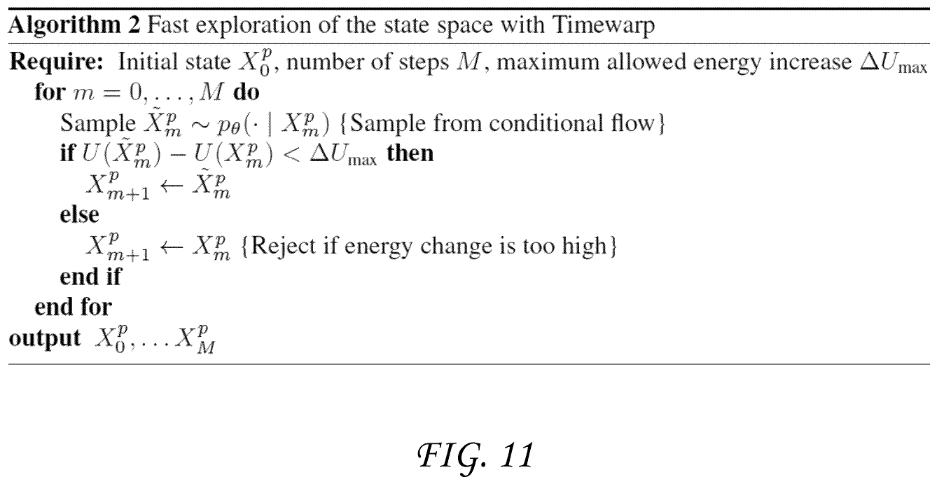

Another application of the MD accelerator, as shown by example in Algorithm 2 of FIG. 11, is to explore the state space. To this end the MD accelerator is used to propose new states and then relax the energy with a few MD steps. If the energy stays too high an update is rejected. More particularly all proposed states of energies above a predetermined cut-off are accepted. The samples generated in this manner will not follow the Boltzmann distribution, but the exploration of the state space is much faster than with MD or with Algorithm 1. As the MD accelerator step can be performed batch wise, it is desirable to generate B chains in parallel, all starting from the same initial state. Parameters for the exploration experiments are B=1000, M=100 and K=50. In some examples, sampling via Algorithm 2 may provide a 10 to 100× speed-up.

The approach here is the same as in the Exploration of Metastable states subsection hereinabove, but without the MD. Instead a proposal is rejected if the energy difference of the proposal is too high. Currently 300 KJ/mol is used as a threshold.

### Proof of Proposition

Provided in this section are more details on the equivariance of the MD accelerator architecture. The proposition will now be proved:

Proof. Let X(t+τ)ₓ₍ₜ₎ denote the random variable obtained by sampling Z˜p(z) and computing X(t+τ):=ƒ(Z;x(t)). The subscript in X(t+τ)ₓ₍ₜ₎ emphasizes that this is the random variable obtained when conditioning the flow on x(t). It will be noted that the equivariance condition on the densities p(σx(t+τ)|σx(t))=p(x(t+τ)|x(t)) is equivalent to the following constraint on the random variables:

$${X ( t + τ )}_{σ x ( t )} {=}^{d} σ {X ( t + τ )}_{x ( t )} , \qquad ( 24 )$$

where [symbol] denotes equality in distribution. To see this, let p_{X} denote the density of the random variable X. Then, in terms of densities, eq 24 is equivalent to stating that, for all x,

$$\begin{matrix}{p}_{{X ( t + τ )}_{σ x ( t )} ( x )} = {p}_{σ {X ( t + τ )}_{x ( t )}} ( x ) & ( 25 ) \\ = {p}_{{X ( t + τ )}_{x ( t )}} ( {σ}^{- 1} x ) , & ( 26 )\end{matrix}$$

where in eq 26 the change-of-variables formula is used, along with the fact that the group actions considered (rotations, translations, permutations) have unit absolute Jacobian determinant. Redefining x←σx, for all x,

$${p}_{{X ( t + τ )}_{σ x ( t )}} ( σ x ) = {p}_{{X ( t + τ )}_{x ( t )}} ( x ) . \qquad ( 27 )$$

Recalling the notation that X(t+τ)x(t) is interpreted as the random variable obtained by conditioning the flow on x(t), this can be written as

$$p ( σ x \vert σ x ( t ) ) = p ( x \vert x ( t ) ) , \qquad ( 28 )$$

which is exactly the equivariance condition stated in terms of densities above. Having rephrased the equivariance condition in terms of random variables in eq 24, the proof of the proposition is straightforward.

$$\begin{matrix}{X ( t + τ )}_{σ x ( t )} := f ( Z , σ x ( t ) ) & ( 29 ) \\ {=}^{d} f ( σ Z , σ x ( t ) ) & ( 30 ) \\ = σ f ( Z , x ( t ) ) & ( 31 ) \\ := σ {X ( t + τ )}_{x ( t )} , & ( 32 )\end{matrix}$$

where eq 30 reflects the fact that the base distribution p(z) is σ-invariant.

### Translation Equivariance via Canonicalization

Described next is the canonicalization technique used to make the models translation equivariant. Let q(x^{p}(t+τ), x^{v}(t+τ)|x^{p}(t)) be an arbitrary conditional density model, which is not necessarily translation equivariant. It can be made translation equivariant in the following way. Let x̅^̅{̅p̅}̅ denote the average position of the atoms,

$$\overline{{x}^{p}} := \frac{1}{N} {\sum }_{i = 1}^{N} {x}_{i}^{p} . \qquad ( 33 )$$

Then, by definition:

$$p ( {x}^{p} ( t + τ ) , {x}^{v} ( t + τ ) \vert {x}^{p} ( t ) ) := q ( {x}^{p} ( t + τ ) - \overline{{x}^{p} ( t )} , {x}^{v} ( t + τ ) \vert {x}^{p} ( t ) - \overline{{x}^{p} ( t )} ) \qquad ( 34 )$$

where the subtraction of x̅^̅{̅p̅}̅(̅t̅)̅ is broadcasted over all atoms. The effect of translating both x^{p}(t) and x^{p}(t+τ) by the same amount is now considered. Let a be a translation vector in [symbol] . Then

$$\begin{matrix}\begin{matrix}p ( {x}^{p} ( t + τ ) + a , \\ {x}^{v} ( t + τ ) \vert {x}^{p} ( t ) + a )\end{matrix} = q ( {x}^{p} ( t + τ ) + a - ( \overline{{x}^{p} ( t ) + a} ) , {x}^{v} ( t + τ ) \vert {x}^{p} ( t ) + & ( 35 ) \\ a - ( \overline{{x}^{p} ( t ) + a} ) ) & \\ = q ( {x}^{p} ( t + τ ) + a - \overline{{x}^{p} ( t )} - a , {x}^{v} ( t + τ ) \vert {x}^{p} ( t ) + & ( 36 ) \\ a - \overline{{x}^{p} ( t )} - a ) & \\ = q ( {x}^{p} ( t + τ ) - \overline{{x}^{p} ( t )} , {x}^{v} ( t + τ ) \vert {x}^{p} ( t ) - \overline{{x}^{p} ( t )} ) & ( 37 ) \\ = p ( {x}^{p} ( t + τ ) , {x}^{v} ( t + τ ) \vert {x}^{p} ( t ) ) . & ( 38 )\end{matrix}$$

Hence p is translation equivariant even if q is not.

As noted above, the methods herein may be tied to a computer system of one or more computing devices. Such methods and processes may be implemented as an application program or service, an application programming interface (API), a library, and/or other computer-program product.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 12 schematically shows a non-limiting embodiment of a computing system 1200 that can enact one or more of the methods and processes described above, or be used to implement the computing systems described above. Computing system 1200 is shown in simplified form. Components of the computing system 1200 may be instantiated in one or more personal computers, server computers, network computing devices, and/or other computing devices.

Computing system 1200 includes a logic processor 1234 volatile memory 1236, and a non-volatile storage device 1238. Computing system 1200 may optionally include a display subsystem 1240, input subsystem 1242, communication subsystem 1244, and/or other components not shown in FIG. 12.

Logic processor 1234 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 1234 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 1238 includes one or more physical devices configured to hold instructions executable by the logic processors to implement the methods and processes described herein. When such methods and processes are implemented, the state of non-volatile storage device 1238 may be transformed—e.g., to hold different data.

Non-volatile storage device 1238 may include physical devices that are removable and/or built in. Non-volatile storage device 1238 may include optical memory, semiconductor memory, and/or magnetic memory, or other mass storage device technology. Non-volatile storage device 1238 may include nonvolatile, dynamic, static, read/write, read-only, sequential-access, location-addressable, file-addressable, and/or content-addressable devices. It will be appreciated that non-volatile storage device 1238 is configured to hold instructions even when power is cut to the non-volatile storage device 1238.

Volatile memory 1236 may include physical devices that include random access memory. Volatile memory 1236 is typically utilized by logic processor 1234 to temporarily store information during processing of software instructions. It will be appreciated that volatile memory 1236 typically does not continue to store instructions when power is cut to the volatile memory 1236.

Aspects of logic processor 1234, volatile memory 1236, and non-volatile storage device 1238 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms ‘module,’ ‘program,’ and ‘engine’ may be used to describe an aspect of computing system 1200 typically implemented in software by a processor to perform a particular function using portions of volatile memory, which function involves transformative processing that specially configures the processor to perform the function. Thus, a module, program, or engine may be instantiated via logic processor 1234 executing instructions held by non-volatile storage device 1238, using portions of volatile memory 1236. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms ‘module,’ ‘program,’ and ‘engine’ may encompass individual or groups of executable files, data files, libraries, drivers, scripts, database records, etc.

When included, display subsystem 1240 may be used to present a visual representation of data held by non-volatile storage device 1238. The visual representation may take the form of a graphical user interface (GUI). As the herein described methods and processes change the data held by the non-volatile storage device, and thus transform the state of the non-volatile storage device, the state of display subsystem 1240 may likewise be transformed to visually represent changes in the underlying data. Display subsystem 1240 may include one or more display devices utilizing virtually any type of technology. Such display devices may be combined with logic processor 1234, volatile memory 1236, and/or non-volatile storage device 1238 in a shared enclosure, or such display devices may be peripheral display devices.

When included, input subsystem 1242 may comprise or interface with one or more user-input devices such as a keyboard, mouse, or touch screen. In some embodiments, the input subsystem may comprise or interface with selected natural user input (NUI) componentry.

When included, communication subsystem 1244 may be configured to communicatively couple various computing devices described herein with each other, and with other devices. Communication subsystem 1244 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 1200 to send and/or receive messages to and/or from other devices via a network such as the Internet.

‘And/or’ as used herein is defined as the inclusive or V, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computerized method for forecasting a future conformation of a molecule based on a current conformation of the molecule, wherein at least the future conformation is a sample drawn from a Boltzmann distribution of conformations of the molecule, the method comprising:
receiving the current conformation in a trained machine-learning model that has been previously trained to map a plurality of conformations received to a corresponding plurality of conformations proposed;
mapping the current conformation to a proposed conformation via the trained machine-learning model, wherein the proposed conformation is appended to a Markov chain;
submitting the proposed conformation to a Metropolis-Hastings test configured to accumulate the Boltzmann distribution without asymptotic bias, by accepting some of the conformations proposed and rejecting a balance of the conformations proposed; and
returning the proposed conformation as the future conformation if the proposed conformation is accepted by the Metropolis-Hastings test.

2. The method of claim 1, wherein the trained machine-learning model maps the conformations received to the conformations proposed by enacting an invertible, normalizing-flow function on a sample drawn from a normal distribution around each conformation received, and wherein the normalizing-flow function is parameterized by learned parameter values of the trained machine-learning model.

3. The method of claim 1, wherein the trained machine-learning model includes one or more transformer blocks adapted to transform vectorized representations of atomic nuclei corresponding to each of the conformations received, and wherein each of the one or more transformer blocks includes a multi-head self-attention mechanism.

4. The method of claim 3, wherein the multi-head self-attention mechanism computes a kernel-weighted self attention where the influence of a first atomic nucleus on a transformation of a second atomic nucleus varies as a function of distance between the first and second atomic nuclei.

5. The method of claim 2, wherein the trained machine-learning model comprises a diffusion model.

6. The method of claim 1, wherein the Markov chain is grown according to a Markov-chain Monte Carlo algorithm.

7. The method of claim 1, wherein rejecting the balance of the conformations proposed comprises pruning the balance of the conformations from the Markov chain.

8. The method of claim 1, wherein each of the conformations received comprises nuclear coordinates on a Cartesian coordinate system or on a coordinate system obtained from the Cartesian coordinate system by a linear transformation.

9. The method of claim 1, further comprising training a machine-learning model to generate the trained machine-learning model, at least in part by:
for each of a plurality of molecules:
subjecting a first conformation of the molecule to a succession of kinematic nuclear displacements to yield a second conformation of the molecule advanced in time by a predetermined interval, wherein each displacement is based on a temperature of the Boltzmann distribution according to a molecular dynamics algorithm, and
coordinately incorporating the first conformation into a first training set and the second conformation into a second training set;
receiving the first and second training sets in the machine-learning model during training; and
adjusting parameter values of the machine-learning model to minimize a residual for mapping elements of the first training set to corresponding elements of the second training set, to thereby generate the trained machine-learning model.

10. The method of claim 9, wherein the molecular-dynamics algorithm approximates integration of differential equations of motion of the molecule over time using a discretizing timestep, and wherein the predetermined interval is at least six orders of magnitude longer than the discretizing timestep.

11. The method of claim 9, wherein the predetermined interval is at least one nanosecond.

12. The method of claim 1, wherein the molecule comprises one or more of an oligopeptide, polypeptide, protein, biomolecule, or polymer.

13. The method of claim 1, wherein receiving and mapping the current conformation and submitting and returning the proposed conformation are enacted repeatedly, such that each future conformation comprises a sample from the Boltzmann distribution.

14. The method of claim 1, wherein receiving and mapping the current conformation and submitting and returning the proposed conformation are enacted repeatedly, the method further comprising:
estimating the rate constant based on a probability that the future conformation approaches the isomeric configuration within the predetermined increment.

15. A computer system for forecasting a future conformation of a molecule based on a current conformation of the molecule, wherein at least the future conformation is a sample drawn from a Boltzmann distribution of conformations of the molecule, the computer system comprising:
a processor and associated computer memory storing a molecular dynamics program that when executed causes the processor to implement:
an input engine configured to receive a primary structure of the molecule;
a generator engine configured to generate the current conformation of the molecule based on the primary structure received;
a molecular dynamics accelerator engine configured to:
receive the current conformation in a trained machine-learning model that has been previously trained to map a plurality of conformations received to a corresponding plurality of conformations proposed;
map the current conformation to a proposed conformation via the trained machine-learning model, wherein the proposed conformation is appended to a Markov chain;
submit the proposed conformation to a Metropolis-Hastings test configured to accumulate the Boltzmann distribution without asymptotic bias, by accepting some of the conformations proposed and rejecting a balance of the conformations proposed; and
return the proposed conformation as the future conformation if the proposed conformation is accepted by the Metropolis-Hastings test; and
an output engine configured to output the future conformation of the molecule as returned by the accelerator engine.

16. The computer system of claim 15, wherein the trained machine-learning model is configured to map the conformations received to the conformations proposed by enacting an invertible, normalizing-flow function on a sample drawn from a normal distribution around each conformation received, and wherein the normalizing-flow function is parameterized by learned parameter values of the trained machine-learning model.

17. A computerized method for forecasting a future conformation of a molecular system based on a current conformation of the molecular system, the method comprising:
receiving the current conformation in a trained machine-learning model that has been previously trained to map a plurality of conformations received to a corresponding plurality of conformations proposed;
mapping the current conformation to a proposed conformation via the trained machine-learning model, wherein the proposed conformation is appended to a Markov chain; and
returning the proposed conformation as the future conformation.

18. The method of claim 17, wherein the molecular system comprises a subset of atoms of a molecule.

19. The method of claim 17, wherein the future conformation corresponds to a metastable state of the molecular system.

20. The method of claim 17, further comprising submitting the proposed conformation to a Metropolis-Hastings test configured to accumulate a Boltzmann distribution without asymptotic bias, by accepting some of the conformations proposed and rejecting a balance of the conformations proposed,
wherein returning the proposed conformation comprises returning only if the proposed conformation is accepted by the Metropolis-Hastings test.
